(19) United States
(12) Patent Application Publication
(10) Pub. No.: US 2014/0065074 A1
(43) Pub. Date: Mar. 6, 2014

(54) METHOD OF TRACKING GROWTH AND METASTASIS OF SPECIFIC CELLS IN VIVO

(71) Applicant: ACADEMIA SINICA, Taipei (TW)

(72) Inventors: Yeu-Kuang Hwu, Taipei (TW); Chia-Chi Chien, Taipei (TW)

(73) Assignee: ACADEMIA SINICA, Taipei (TW)

(21) Appl. No.: 14/016,061

(22) Filed: Aug. 31, 2013

(30) Foreign Application Priority Data
Aug. 31, 2012 (TW) 101131690

(51) Int. Cl. A61K 49/04

(52) U.S. Cl. CPC A61K 49/04
USPC 424/9.4

(57) ABSTRACT

A method of tracking growth and metastasis of specific cells in vivo is disclosed. The method of the disclosure includes culturing the specific cells in a medium containing 0.1 μM to 10 mM nanoparticles as a biomarker for X-rays, such that the specific cells carry the nanoparticles, administering the specific cells carrying the nanoparticles to a subject, irradiating the subject by an X-ray source, and determining the growth and metastasis of the specific cells by X-ray images of the nanoparticles in the subject.

### CROSS REFERENCE TO RELATED APPLICATIONS

This application claims priority of Taiwan Patent Application No. 101131690 filed on Aug. 31, 2012, the entirety of which is incorporated by reference herein.

### BACKGROUND OF THE INVENTION

1. Field of the Invention

The present invention relates to a method of tracking cells in vivo, and in particular, relates to a method of tracking growth and metastasis of specific cells in vivo using X-ray absorbed nanoparticles as biomarkers for specific cells.

2. Description of the Related Art

Gold nanoparticles (Au-NPs) have been used in a variety of nanotechnology, such as bio-sensing, biological imaging, and nanoscale treatment. Au-NPs play an important role in the biomedical fields such as health, diagnosis, and fighting malignant diseases such as cancer. Au-NPs are small in size and have Enhanced Permeability and Retention Effect (EPR) in tumor parts, and are able to agglomerate in cancer tissues with high selectivity. Therefore, Au-NPs are suitable as drug delivery carriers or radiotherapy enhancers.

The mechanism for carrying Au-NPs by cells and the amount of carried Au-NPs are affected by the size, surface properties, colloid stability and other properties of the Au-NPs. In the past, there were many studies using Au-NPs as biomarkers in vivo, and Au-NPs were modified by various kinds of methods to comply with different clinical research needs. In fact, Au-NPs without surface modification can also be swallowed by cells. However, influenced by the way Au-NPs are synthesized, the amount of the Au-NPs that can be carried by cells is limited by the physical and chemical properties of the produced Au-NPs. In Phys. Med. Biol. 49 (2004) N309, Hainfeld used a high concentration of the Au-NPs (1.9 nm) to conduct animal radiotherapy experiments.

Although nanomaterials have already been developed, there are still many difficulties in applying nanomaterials to the imaging of tumors or specific cells in vivo. The imaging of tumor cells plays an important role in clinical practice.

In view of this, a more effective method of tracking growth and metastasis of tumor cells is needed to provide a more accurate clinical diagnosis tool.

### BRIEF SUMMARY OF THE INVENTION

A detailed description is given in the following embodiments with reference to the accompanying drawings.

A method of tracking growth and metastasis of specific cells in vivo is disclosed. The method of the disclosure includes: culturing the specific cells in a medium containing 0.1 μM to 10 mM of nanoparticles as a biomarker for X-rays, such that the specific cells carry the nanoparticles; administering the specific cells carrying the nanoparticles to a subject; irradiating the subject by an X-ray source; and determining the growth and metastasis of the specific cells by X-ray images of the nanoparticles in the subject.

### BRIEF DESCRIPTION OF THE DRAWINGS

The present invention can be more fully understood by reading the subsequent detailed description and examples with references made to the accompanying drawings, wherein:

FIGS. 1A-1C show the uptake of the Au-NPs by the EMT-6 cells in accordance with some embodiments of the present disclosure;

FIG. 1D and FIG. 1E show the growth rate of cells with or without Au-NPs.

FIG. 2A-2H show the growth of the EMT-6 cells inoculated in the subcutaneous tissue of the left leg region of mice in accordance with some embodiments of the present disclosure;

FIGS. 3A-3E show optical pathological images of a subcutaneous tumor induced by shallow inoculation of tumor cells carrying Au-NPs;

FIG. 3F shows a nano resolution TXM projected image of Au-NPs in cells;

FIG. 3G shows a tomographic reconstructed image of the square area of FIG. 3F;

FIGS. 3H-3I show tomographic reconstructed images of a part of the area in FIG. 3F;

FIG. 3J shows a nano resolution TXM image, which is a block micrograph corresponding to the area marked by the straight line in FIG. 3E;

FIGS. 3K-3N are nano resolution TXM projected images of Au-NPs in tissue corresponding to the site of FIG. 3J;

FIGS. 4A and 4B are projective X-ray images with lower and higher magnification, demonstrating images of a lung tissue with undyed Au-NPs in a formaldehyde solution after the mice have been inoculated for 7 days;

FIGS. 4C-4D are nano resolution X-ray (TXM) images, demonstrating images of lung cancer with Au-NPs carried by CT-26 colon cancer cells;

FIG. 4E is an optical image of lung tissue with H&E staining;

FIGS. 4F and 4G are respectively a single projective image and a tomography reconstructed image, in which Au-NPs with a particle size of less than 60 nm were observed in the tumor cells; and

FIGS. 4H-4J are different slices of tomographic reconstructed images of FIG. 4G

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

The present disclosure provides a method of co-culturing specific cells and bare nanoparticles as a biomarker for X-rays, such that the specific cells carry a satisfactory amount of nanoparticles. The specific cells carrying the nanoparticles are administered to a subject and the subject is irradiated by an X-ray source. The growth and metastasis of the specific cells are determined by X-ray images of the nanoparticles in the subject. The method of co-culturing specific cells and bare nanoparticles as a biomarker for X-rays includes co-culturing the specific cells in a medium containing about 0.1 μM to 10 mM, preferably 0.5 mM, of nanoparticles as a biomarker for X-rays for 24 hrs., preferably 12 hrs.

The X-ray source may be a high-energy and high-dose (4 keV-20 MeV) X-ray source with high penetration ability. In one embodiment, the X-ray source may be a synchrotron radiation X-ray source.

The high penetration ability of the high-dose X-ray source (about 700-850 nm of wavelength) overcomes the inadequate penetration of photons in vivo, and is able to efficiently stimulate the nanoparticles as a biomarker administrated in the subject. In addition, since the dosage of X-ray source is high enough, irradiation may be performed for less than about 1 second, preferably less than about 100 milliseconds. The effective penetration depth of the subject irradiated by the X-ray source may be about 30 cm from the surface to the deep tissue. Since the high-energy X-ray source adopted in the present disclosure has a high penetration ability in vivo, tumor cells in vivo may be monitored immediately by X-ray imaging of the present disclosure, instead of having to perform sample slicing from living subjects as conventional medical imaging requires.

The present disclosure is suitable for tracking any kinds of somatic cells in a subject, such as tumor cells, wherein the subject may include humans, mammals, birds, amphibians, reptiles, fish, insects, and/or other appropriate multicellular animals.

In one embodiment, methods of administering the specific cells carrying the nanoparticles to a subject may include, but are not limit to, subcutaneous injection, intraperitoneal injection, intramuscular injection, intravenous injection, arterial injection, lymphatic injection, and/or local organ injection.

The present disclosure providing bare nanoparticles as X-ray biomarkers may include Au-NPs with good stability and biocompatibility.

Using 3D tomographic reconstruction (−1 μm) and a zone plate full-field transmission hard-x-ray microscope (TXM) to image high-resolution X-ray images (−15 nm), the growth and metastasis of specific cells in vivo may be efficiently tracked.

### EXAMPLES

### Example 1

### Cell Culture

EMT-6 breast cancer cells and CT-26 colon cancer cells were obtained from the American Type Culture Collection (ATCC) and cultured at 37° C. in humid air with 5% CO₂. The EMT-6 breast cancer cells were incubated with the Dulbecco's Modified Eagle's Medium: Nutrient Mixture F-12 (DMEM/F12)/10% fetal calf serum (FCS). CT-26 colon cancer cells were incubated with RPMI-1640/10% FCS. All media were supplied by Gibco.

### Example 2

### Cell Proliferation Tests

EMT-6 breast cancer cells were co-cultured with 500 μM of gold nanoparticles (Au-NPs) for 24 hrs., after a trypsin treatment. The EMT-6 breast cancer cells with and without Au-NPs were separately seeded on a culture dish, wherein the cells were continuously cultured and the cell number was counted every two days. The cell counting results for cells exposed and unexposed (control cells) were compared to assess the effects of the nanoparticles on proliferation of the EMT-6 breast cancer cells. The results show equivalent rates of cell proliferation of the EMT-6 breast cancer cells with and without Au-NPs.

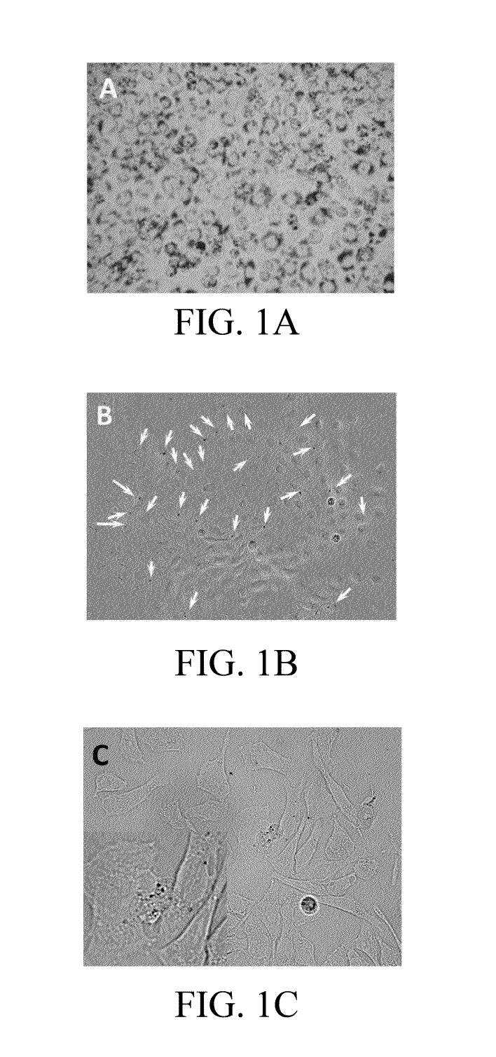

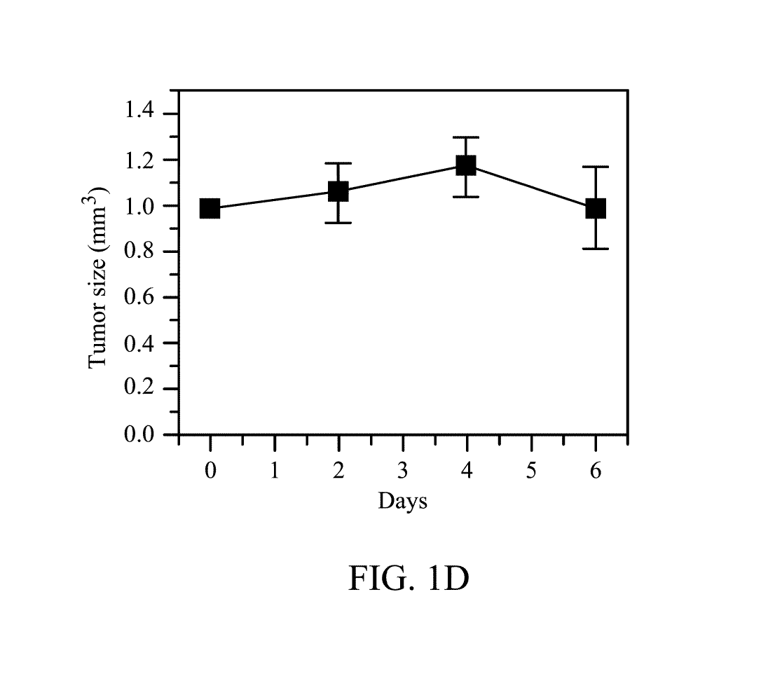

FIGS. 1A-1D show the uptake of the Au-NPs by the EMT-6 cells in accordance with some embodiments of the present disclosure. As shown in FIG. 1a, a large number of the Au-NPs were located in the cytoplasm after 1 day co-culturing of the Au-NPs and EMT-6 cells. After 6 days and several cell cycles, the Au-NPs were still present in the cells although the number of the Au-NPs per cell decreased (FIG. 1B). The optical image of FIG. 1C shows cells in the mitotic phase, indicating that the Au-NPs passed from the first cell generation to the next generation. The growth rate of cells with Au-NPs was similar to that of the control cells (not co-cultured with Au-NPs), as shown in FIGS. 1D and 1E, indicating that the Au-NPs had good biocompatibility.

### Example 3

### Inoculation of Tumor Cells

The EMT-6 breast cancer cells and CT-26 colon cancer cells were co-cultured with 500 μM of the Au-NPs for 24 hrs., after a trypsin treatment. Then, harvested cells were added to the PBS. 50 μl of a 1×10⁷ cells/ml EMT-6 breast cancer cell solution were inoculated in the subcutaneous tissue of the left leg region of mice. Meanwhile, 100 μl of a 1×10⁷ cells/ml of the CT-26 colon cancer cell solution, were introduced by tail vein injection into the mice. The inoculated EMT-6 breast cancer cells and CT-26 colon cancer cells were preliminary tumor cells. After inoculating the preliminary tumor cells, two kinds of cells developed into tumors. The development depended upon the inoculation method. The size of tumor was 100-120 mm³ after 7 days. The subcutaneous tumor volume was estimated with the formula v=0.5×a×b², where a and b were the smallest and the largest diameters, respectively.

The mice used in this example were BALB/cByJNarl mice (purchased from the National Laboratory Animal Center, Taiwan) approved by the Academia Sinica Institutional Animal Care and Utilization Committee (AS IACUC). All mice were housed in individual cages (five per cage) and kept at 24±2° C. with a humidity of 40%-70% and a 12-hour light/dark cycle.

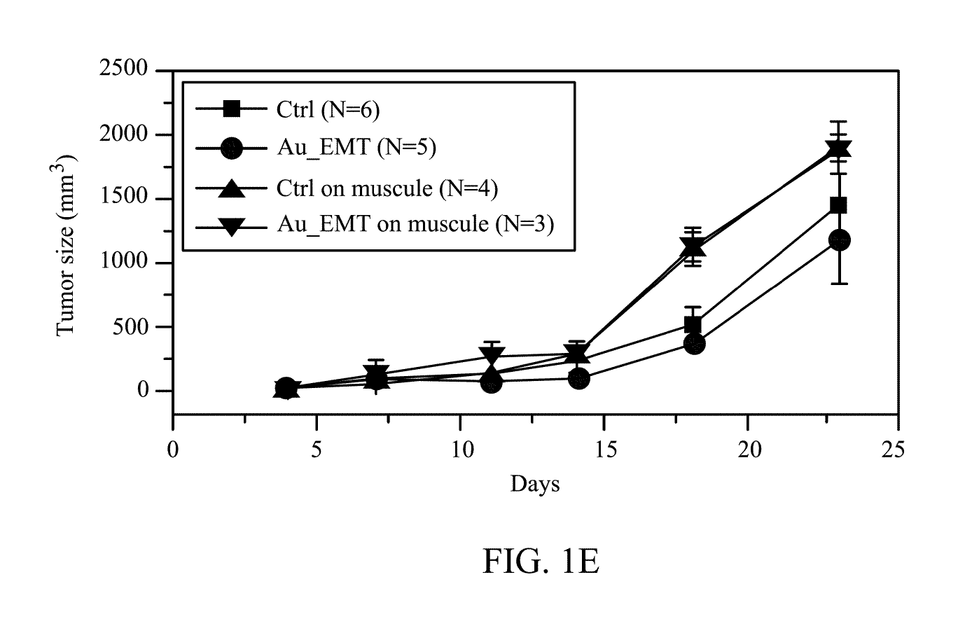

FIG. 2A-2H show the growth of the EMT-6 cells inoculated in the subcutaneous tissue of the left leg region of mice in accordance with some embodiments of the present disclosure. FIG. 2A shows an image of the 3 day developed tumor after the inoculation of the EMT-6 cells. FIG. 2B shows a magnified image of FIG. 2A. As shown in FIGS. 2A and 2B, when the thigh tumor development reached day 3, a complete vessel network was already initiated for tumor growth. In FIG. 2B, the small arteries supplying oxygen were marked by arrows and red vessels, and the reflowing small veins were marked by arrows and blue vessels. FIG. 2C shows an image of 7 day developed tumor after the inoculation of the EMT-6 cells, and FIG. 2D and FIG. 2E are tomographic reconstructed images of FIG. 2C with lower and higher magnification. FIG. 2F shows a projective image of the agiogenesis of the 7 day developed tumor after the inoculation of the EMT-6 cells without Au-NPs. FIG. 2G and FIG. 2H are tomographic reconstructed images of FIG. 2F with lower and higher magnification. As shown in FIGS. 2F, 2G, and 2H, the angiogenesis of the 7 day tumor became richer than the 3 day tumor. Also, according to each image of FIG. 2, most of the inoculated preliminary tumor cells stayed at the inoculated site due to cell apoptosis, and angiogenesis developed around and failed in the central area of the necrotic region. The scale bar in each image of FIG. 2 was 500 μm.

### Example 4

### Real Time X-Ray Imaging

Microscopic X-ray imaging was implemented with the irradiation of a BL01-A beamline (Margaritondo G, Hwu Y and Je J H 2004 Rivista del Nuovo Cimento 27 7) in a storage ring of the National Synchrotron Radiation Research Center (NSRRC) (Hsinchu, Taiwan). The beamline energy ranged from 4 keV to 30 keV with a central energy of about 12 keV and the beam current was kept constant at 360 mA. Images obtained in this example were 4.5×3.4 mm, and the synchrotron radiation X-ray sources were first converted to visible light by a CdWO₄ single crystal scintillator and then captured by an optical image with a CCD camera (model 211, Diagnostic instruments, 1600×1200 pixel). Before irradiating the subjects by the synchrotron radiation X-ray sources, the radiation dose was reduced by attenuating the emitted X-ray beam with two pieces of 550 μm single crystalline silicon wafers. During X-ray irradiation, the mice were kept under anesthesia using 1% isoflurene in oxygen. The exposure time was about 100 milliseconds, and the distance between the sample and the scintillator was about 5 cm.

### Example 5

### High-Resolution X-Ray Imaging

In this example, the high-resolution micro X-ray imaging was performed on the 32-ID microscopy beamline of the Advanced Photon Source (APS) in the Argonne National Laboratory. The zone plate full-field x-ray transmission microscope (TXM) used a set of capillary condensers to precisely illuminate the subjects. The condensers were elliptically shaped glass capillaries. The inner diameter of 0.9 mm was chosen to maximize the vertical acceptance of the Advanced Photon Source (APS) undulator beam at 65 m from the source. In this example, the monochromatic X-ray flux was estimated by a Si (111) double crystal monochromator focused by the condenser. The estimated monochromatic X-ray flux was 2×10¹¹ photons/s at 8 keV. The high brightness of the APS and the optimized condensers design yielded an excellent imaging rate of 50 ms/frame with −1×10⁴ CCD (charge coupled device) counts per pixel.

The microscope system also operated in the Zernike phase contrast imaging mode with an Au phase ring placed at the back focal plane of the FZP objective.

### Example 6

### Tissue Sample

After inoculation with tumor cells by subcutaneous and tail vein injections for one week, mice (about 20-25 g of weight) were sacrificed by an overdose of Zoletil 50 (50 mg/kg; Virbac Laboratories, Carros, France) by intramuscular injection, to remove subcutaneous tissues and lungs. Tissue specimens were immersed in the 3.7% paraformaldehyde for 24 hr. After fixation, the tissues were washed by PBS (1× phosphate buffer solution) three times per 1 hr. Tissues were separated into two groups, one for micro and the other for nano resolution X-ray imaging. Micro resolution X-ray tomography imaging required thick tissues (about 30 μm) embedded in resin. Nano resolution X-ray imaging required specimens embedded in paraffin. All tissues were dehydrated by subsequent immersions in ethanol solutions, from low to high concentration, and then embedded in the resin or paraffin. Such specimens were immersed in Xylene for three times for 5 minutes each to remove the remaining wax. Afterwards, the specimens were dehydrated with the same procedure described above and immersed in the distilled water.

Some of the specimens were stained with H&E for optical microscopy, and others with osmium tetroxide staining for X-ray imaging. The stained specimens were washed with distilled water 3 times for 5 minutes each, dehydrated as above and embedded in an Embed-812 Resin (EMS, Hatfield, Pa.).

### Example 7

### Tomography

Thick samples (about 30 μm) in resin from Example 6 were used to take sets of 1000 images within 180 degrees. Tomographic reconstruction was then performed by Interactive Data Language (IDL) software. All reconstructed images were processed with the Amira 5.2 software to obtain three dimensional pictures.

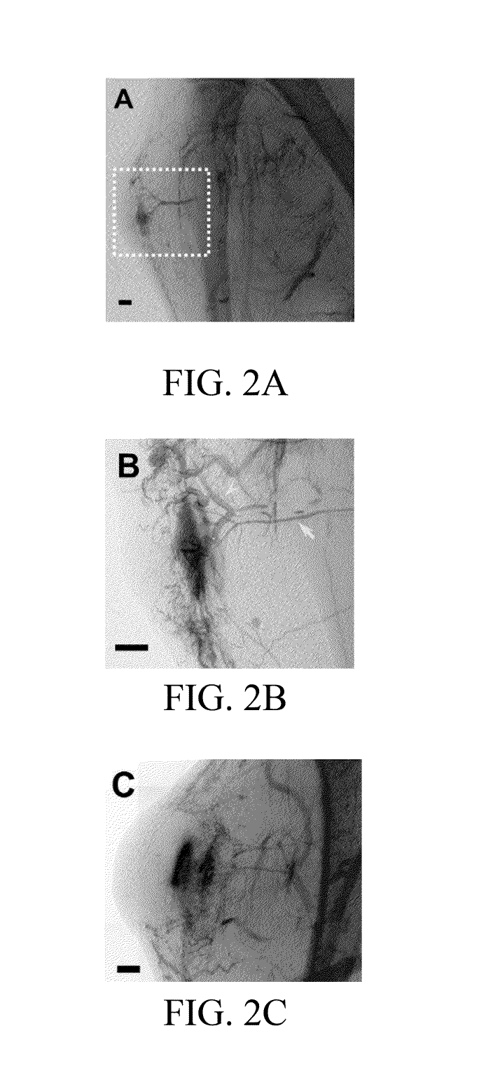

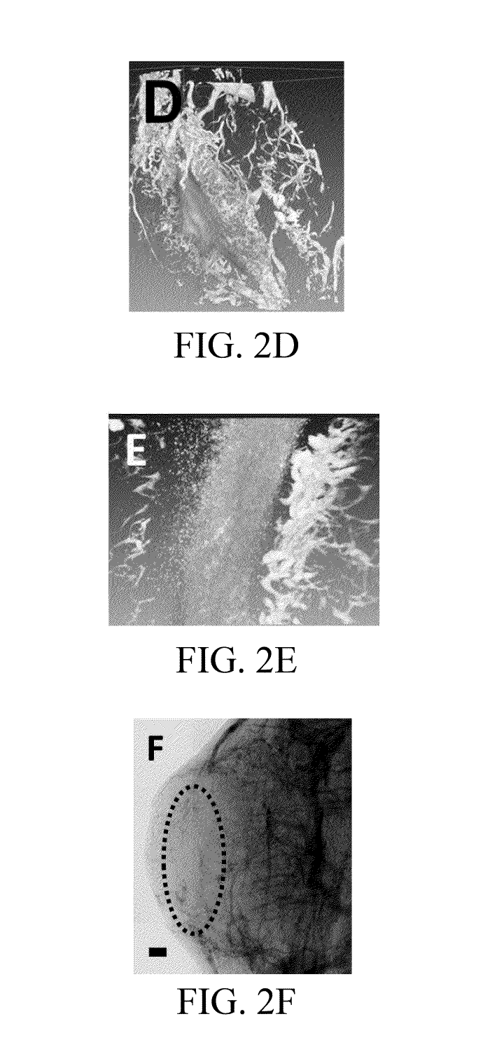

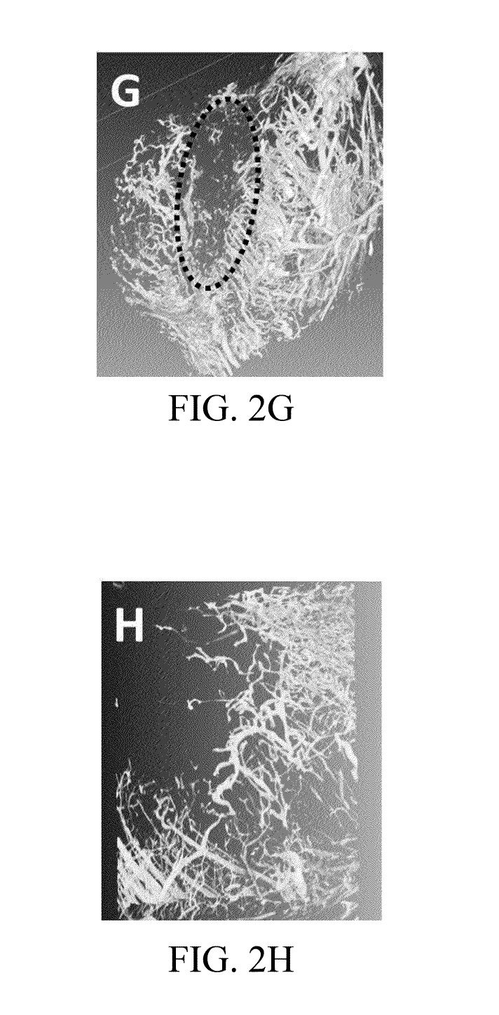

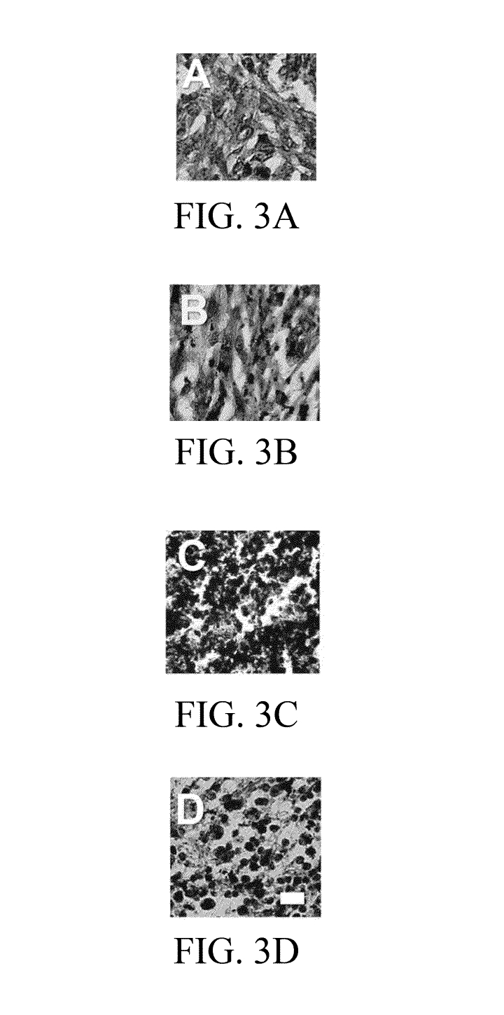

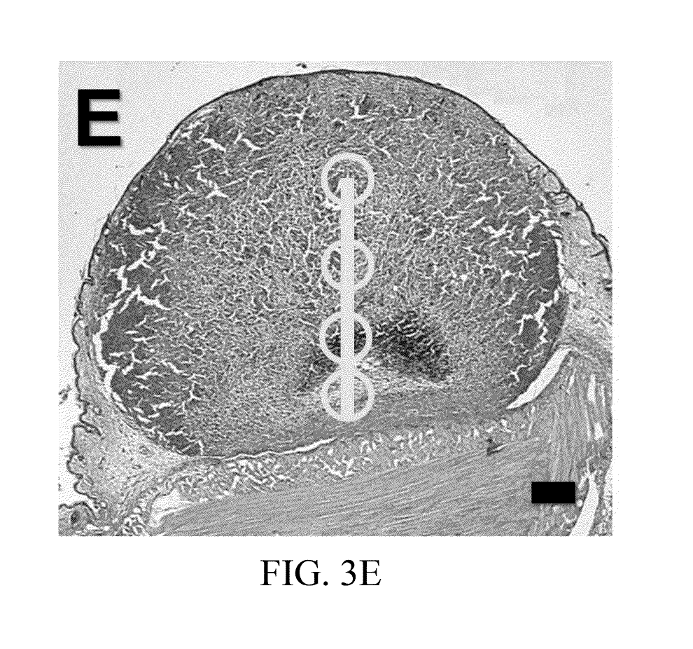

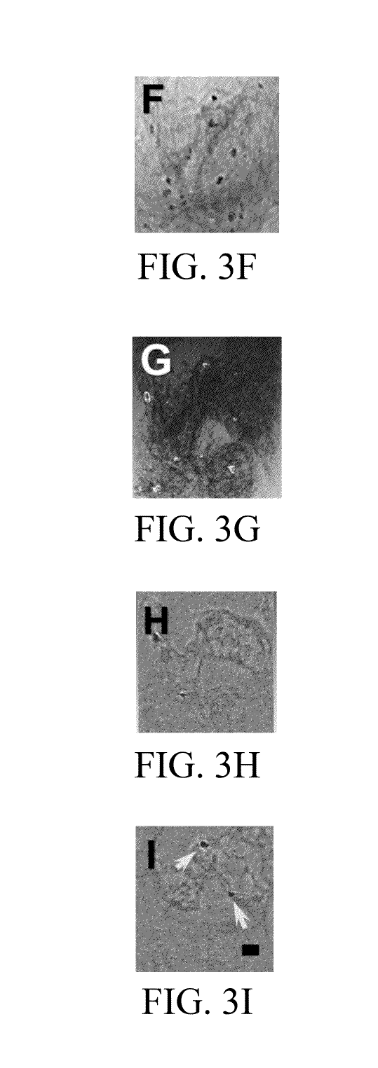

FIGS. 3A-3E show optical pathological images of a subcutaneous tumor induced by shallow inoculation of tumor cells carrying Au-NPs. FIGS. 3A-3D demonstrate magnified images of different regions in FIG. 3E. The scale bars in FIGS. 3A-3D were 20 μm, while in FIG. 3E were 200 μm. FIG. 3J shows a nano resolution TXM image, which is a block micrograph corresponding to the area marked by the straight line in FIG. 3E. FIG. 3F is a nano resolution TXM projected image which show cell-carried Au-NPs in tumor tissue.

FIG. 3G shows a tomographic reconstructed image of the square area of FIG. 3F. FIGS. 3K-3N show different areas of FIG. 3J, which have gredient concentration of Au-NPs in tumor. The scale bars in FIGS. 3K-3N were 5 μm, while in FIG. 3J were 50 μm. FIGS. 3H-3I show tomographic reconstructed images of a part of the area in FIG. 3F. Highlights shown in FIG. 3G are the aggregation of the Au-NPs.

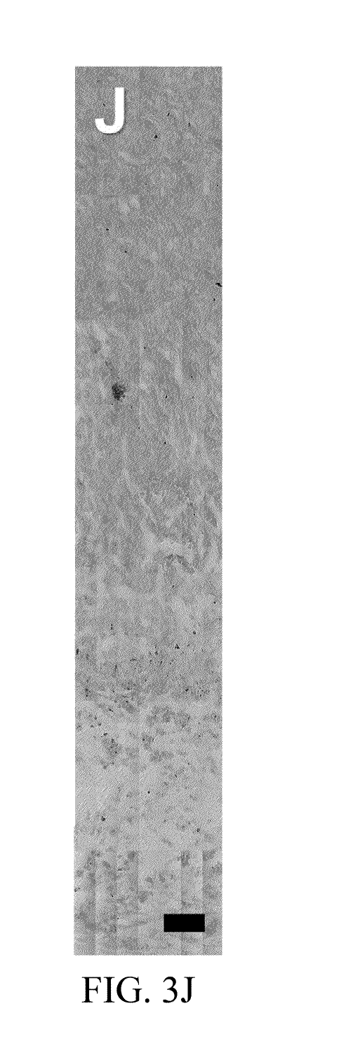

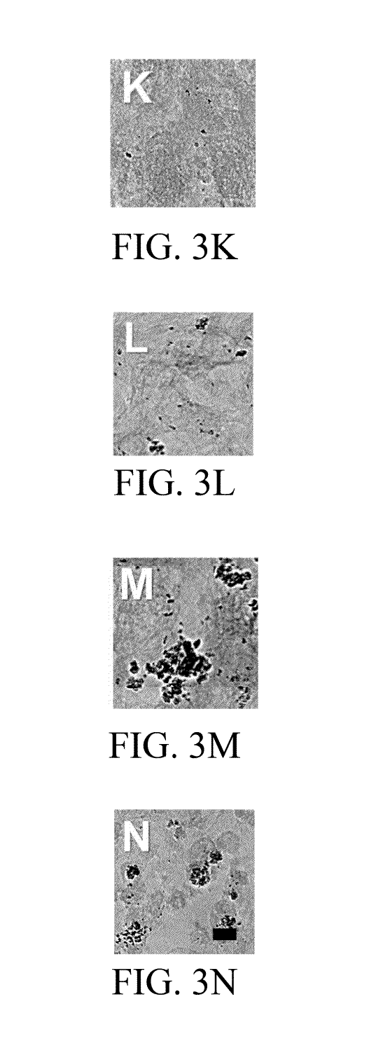

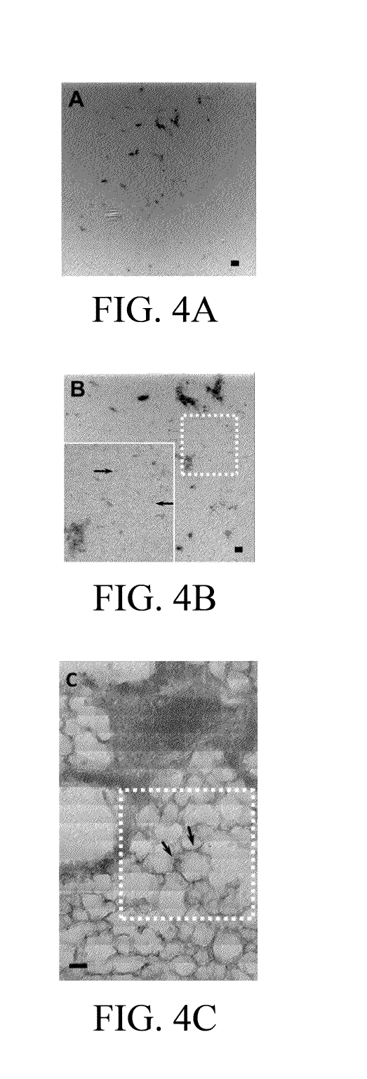

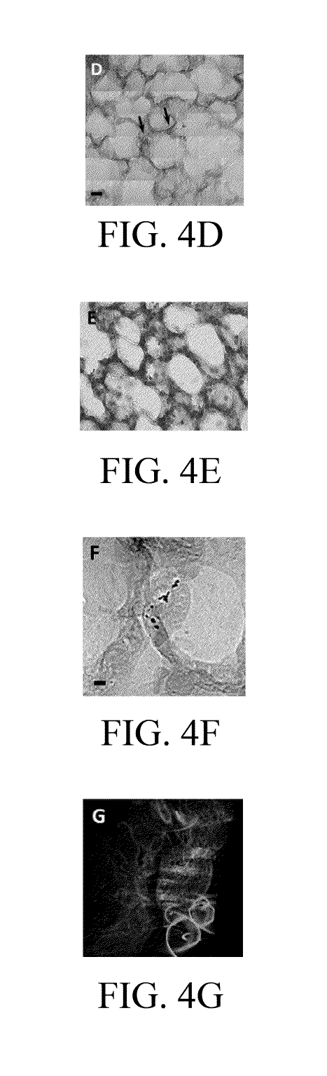

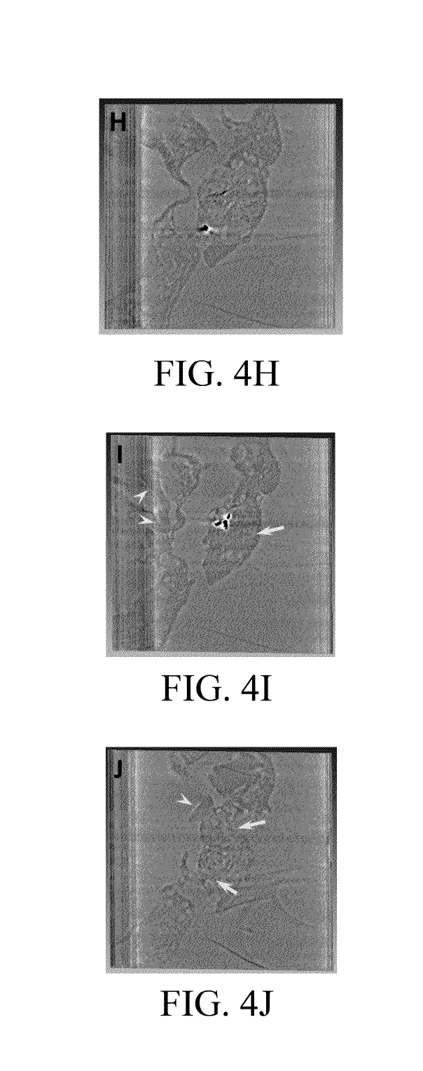

FIGS. 4A and 4B are projective X-ray images with lower and higher magnification, demonstrating images of a lung tissue with undyed Au-NPs in a formaldehyde solution after the mice have been inoculated for 7 days. The black dots in FIGS. 4A and 4B are a metastasis model of CT-26 colon cancer cells in lungs. Some cell pellets plugged capillaries and most of the Au-NPs labeled tumor cells were distributed uniformly in the lung tissues. Au-NPs with a particle size of about 3 μm were observed in FIG. 4B indicated by the black arrows. FIGS. 4C-4D are nano resolution X-ray (TXM) images, demonstrating images of lung tumor tissues with Au-NPs carried by CT-26 colon cancer cells. FIG. 4C are patch images of sliced lung tissues, demonstrating the sites (indicated by the black arrows) of tumor cells in the lung tissues. FIG. 4D is a magnified image of FIG. 4C. It was difficult to observe the images of the Au-NPs carried by the tumor cells in the above optical pathologic images. FIG. 4E is an optical image of lung tissue with H&E staining. FIGS. 4F and 4G are respectively a single projective image and a tomography reconstructed image, in which Au-NPs with a particle size of less than 60 nm were observed in the tumor cells (black dots in FIG. 4F, red parts in FIG. 4G). FIGS. 4H-4J are different slices of tomographic reconstructed images of FIG. 4G which show that cancer cells were near the capillary, and erythrocytes (indicated by yellow arrows) flowed through the capillary, and three tumor cells stayed in the alveolar tissue, demonstrating that tumor cells migrated from the nearest capillary wall and formed tumor nodules. The scale bars in FIGS. 4A and 4B were 50 μm and 25 μm, respectively. The scale bars in FIGS. 4C and 4D were 24 μm and 12 μm, respectively. The scale bar in FIG. 4F was 1.7 μm.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method of tracking growth and metastasis of specific cells in vivo, comprising:
culturing the specific cells in a medium containing about 0.1 μM to 10 mM of nanoparticles as a biomarker for X-rays, such that the specific cells carry the nanoparticles;
administering the specific cells carrying the nanoparticles to a subject;
irradiating the subject by an X-ray source; and
determining the growth and metastasis of the specific cells by X-ray images of the nanoparticles in the subject.

2. The method of tracking growth and metastasis of specific cells in vivo as claimed in claim 1, wherein the X-ray source comprises a synchrotron radiation X-ray source, a medical X-ray source, or a laboratory X-ray source.

3. The method of tracking growth and metastasis of specific cells in vivo as claimed in claim 1, wherein the X-ray source has a dose of less than about 100 Gy.

4. The method of tracking growth and metastasis of specific cells in vivo as claimed in claim 1, wherein the dose of the X-ray source is between about 1 Gy and 30 Gy.

5. The method of tracking growth and metastasis of specific cells in vivo as claimed in claim 1, wherein the irradiation is performed for less than 30 minutes.

6. The method of tracking growth and metastasis of specific cells in vivo as claimed in claim 1, wherein the irradiation is performed for between about 100 milliseconds and 5 minutes.

7. The method of tracking growth and metastasis of specific cells in vivo as claimed in claim 1, wherein the subject comprises humans, mammals, birds, amphibians, reptiles, fish, insects, or other appropriate multicellular animals.

8. The method of tracking growth and metastasis of specific cells in vivo as claimed in claim 1, wherein the specific cells comprise tumor cells, stem cells, blood cells, tissue cells, or other appropriate somatic cells.

9. The method of tracking growth and metastasis of specific cells in vivo as claimed in claim 1, wherein the effective penetration depth of the subject irradiated by the X-ray source is about 30 cm from the surface to the deep tissue.
